(19) United States
(12) Patent Application Publication
(10) Pub. No.: US 2011/0064818 A1
(43) Pub. Date: Mar. 17, 2011

(54) PHARMACEUTICAL COMPOSITION AND A METHOD FOR THE PRODUCTION THEREOF

(76) Inventors: David Anatol'evich NOGA, Kiev (UA); Pavel Gueorguievich MATVIEEV, Kiev (UA); Serguei Serqueevich MARKIN, Moscow (RU); Dmitrij Borisovich BERENSHTEIN, Moscow (RU); Mikhail Pietrovich SIEMIENOV, Moscow (RU); Alexandr Andreevich TARASOV, Kiev (UA); Ol'ga Maratovna TARASOVA, Kiev (UA); Igor Viacheslavovich RED'KIN, Kiev (UA)

(21) Appl. No.: 12/883,984

(22) Filed: Sep. 16, 2010

Related U.S. Application Data
(63) Continuation of application No. PCT/UA2008/000019, filed on Mar. 25, 2008.

(30) Foreign Application Priority Data
Mar. 19, 2008 (RU) A 2008 03496

(51) Int. Cl. A61K 9/14; A61K 38/28; B82Y 5/00

(52) U.S. Cl. 424/499; 514/5.9; 977/906

(57) ABSTRACT

The invention relates to producing medicinal agents for treating diabetes. There is proposed a method for producing a pharmaceutical composition containing insulin on a polysaccharide carrier, which involves mixing initial ingredients, wherein there is provided, for mixing, supplying positively charged chitosan sol with pH of 3.5 to 4.5 and negatively charged zinc free insulin, which is taken in the form of a colloidal solution or in the form of nanosized crystalline particles, bringing the pH of the mixed sol to a value of 5.5 to 6.5, producing a gel and dehydrating the produced gel to obtain solid particles, the size of which ranges from 10 to 100 mkm. The method makes it possible to produce a stable controlled release insulin-containing composition for peroral administration

### CROSS-REFERENCE TO RELATED APPLICATIONS

This application is a continuation application of international application PCT/UA 2008/000019 filed on Mar. 25, 2008, which in turn claims priority to Ukrainian application a 2008 03496, filed on Mar. 19, 2008, both of which are incorporated herein by reference.

### FIELD OF THE INVENTION

The invention relates to producing medicinal agents comprising an active operating substance, bound to the carrier, and can be used for obtaining preparations with controlled release of insulin.

### BACKGROUND OF THE INVENTION

Recently the great attention has been given to the development of the pharmaceutical preparations providing for controlled release of operating substance, selecting carriers for active medicinal agents and developing methods for producing preparations in a medicinal form to be suitable for a patient administration.

Insulin is one of the active medicinal substances, which such developments are especially necessary for.

There is known a method for producing a stable dry powdery insulin-containing composition for peroral administration, which comprises the step of dissolving insulin and a pharmaceutical carrier in a water buffer followed by spray-drying the solution to obtain amorphous particles, the average size of which ranges from 0.1 to 10 mkm and the moisture content is less than 10%, thus as a carrier there are used carbohydrates from the group consisting of mannitol, raffinose, lactose, maltodextrin, or sodium organic salts such as citrate, gluconate, ascorbate (RU 2175556, Feb. 20, 1999).

However the obtained powdery agent is only intended for adsorbing insulin through lungs.

There is known a composition for insulin nasal administration wherein as a carrier it is used aggregated crystalline cellulose with specific granulometric distribution (RU 2299743, May 27, 2007).

There is known a pharmaceutical composition for peroral administration containing insulin on polyethylene oxide carrier of molecular mass ranging from 0.4 kDa to 40 kDa. The carrier is preliminary irradiated with a high-energy irradiation, and then insulin is added thereto to obtain its protein concentration of 1 mg/ml to 10 mg/ml and polyethylene oxide—insulin ratio of (1-500:1) and the mixture is agitated to obtain the transparent or slightly opalescence solution (RU 2316339, Feb. 10, 2008).

However the obtained preparation has no property of controlled release of active substance.

To produce medicinal forms of slowed down release, polymeric bioblastable substances are considered the most perspective carriers.

For example, there is known a bioblastable three-block polymer with average molecular mass of 2000-4990 Dalton possessing properties of a reversible thermal gelatinization, that is, existing in the form of an aqueous solution at low temperatures, and forming gel at physiologically suitable temperatures. The polymer contains bioblastable hydrophobic polyester to be prepared on the basis of various monomers as well as a bioblastable hydrophilic polymeric block of polyethylene glycol wherein it is possible to introduce insulin (RU 2232779, Jul. 20, 2004).

There is also known a method to produce microparticles for slowed down release of substance wherein a composition containing an active agent is dispersed in a polymer organic solution, and an obtained mixture is added into external phase in the form of aqueous solution or oil phase, then the solvent is removed under pressure or in a flow of an inert gas. Thus the composition comprising the active agent can contain chitosan with deacylation degree of 25 to 100% and molecular mass of 10000 to 2000000 Da, which is dissolved in acetic acid. As a result of the method realization, there could be obtained spherical microparticles with diameter of 1 to 500 mkm. (RU 2291686, Jan. 20, 2007).

There is known a method wherein a chitosan derivative is chosen taking into account the type and the number of its charged groups, the form of operating agent is also chosen so that at certain pH level, there was provided matching the charges of the operating agent and the carrier in the preparation. From the chosen chitosan derivative, there is obtained water sol containing operating agent, then the water sol pH value is regulated to achieve its isoionic points (condition) with possible sedimentation of colloidal particles or nanosized operating agent, and the obtained water sol is subjected to drying (RU 2256440, Oct. 10, 2003).

In spite of the fact that the list of the active agents, which are suitable to be used in above said methods, includes insulin, the conditions, which are necessary to obtain a preparation with the slowed down release of insulin for peroral administration, are not disclosed in the given patents. The methods known from the art only remind about the possibility of obtaining nanosized particles for operating agent on a chitosan derivative carrier.

As a prototype for the claimed invention, there is proposed a method of producing a pharmaceutical composition containing insulin on a polysaccharide carrier. The method comprises mixing initial ingredients such as a suspension of chitosan in a phosphatic buffered solution with pH=7.3-7.4 or with pH=4 and sodium insulin in a phosphatic buffered solution with pH=7.3-7.4 or with pH=4, the solutions are admixed at volume ratio 1:1, and introducing hydrochloric acid up to obtaining an insulin and chitosan soluble complex with pH=3.6-4.4. The proposed composition is intended for nasal administration or as a vaccine (U.S. Pat. No. 5,554,388, Sep. 10, 1996).

Disadvantage of the known method is the absence of stability that results in obtaining a composition with a wide scatter of properties. The method is also not suitable for obtaining the medicinal form for peroral administration that is characterized by controlled release of insulin.

Perorally entered preparations pass through esophagus, stomach and intestine (the organs having various acidity of medium and various enzymes) that complicates developing effective means for peroral administration. At peroral administration, insulin is split into amino acids and small peptides which are quickly decayed in epithelium of small bowel by aminopeptidase that weakens action of a preparation and reduces efficiency of controlled release.

### BRIEF DESCRIPTION OF THE DRAWING FIGURE

FIG. 1 is a graph showing released insulin over hours.

### DETAILED DESCRIPTION OF THE PREFERRED EMBODIMENT

The technical problem of the present invention is to develop a method for producing insulin on a carrier for peroral administration providing for production of a pharmaceutical composition of slowed down release, which is characterized by the stability of the properties.

The set technical problem is solved realizing the described method for producing a pharmaceutical composition containing insulin on a polysaccharide carrier, which involves mixing initial ingredients being as positively charged chitosan sol with pH of 3.5 to 4.5 and negatively charged zinc free insulin, which is taken in the form of a colloidal solution or in the form of nanosized crystalline particles, bringing the pH of the mixed sol to a value of 5.5 to 6.5, producing a gel and dehydrating thus produced gel to obtain solid particles, the size of which ranges from 10 to 100 mkm.

Preferably, the step of producing gel is performed at presence of sodium alginate in the mixed sol in the amount not exceeding 5% of masses, if calculated per solid substance.

It is provided the step of mixing initial ingredients to be carried out based on calculation of insulin-chitosan mass ratio in the mixed sol, which is equal to (0.2-0.8):1, under condition of the mixed sol electrical neutrality.

Preferably, while mixing, there is supplied a positively charged chitosan sol in a buffered acetate solution with the size of chitosan particles of 200 to 400 nanometers.

Preferably, while mixing, there is supplied a positively charged chitosan sol in the form of its chlorohydrate having molecular mass of 80 to 120 kDa and deacetylation degree of 85 to 89%.

While mixing, there is supplied insulin in the form of crystalline particles, the sizes of which are ranged from 800 to 1200 nanometers or as a colloidal solution of insulin in the phosphatic buffer with pH of 8.0 to 9.0.

The most preferably, while mixing there is supplied chromatography cleaned gene-engineered insulin obtained with the use of STRAIN ESCHERICHIA COLI XLI-BLUE/PINSR as PREPROINSULIN PRODUCENT.

The set technical problem is also solved with the help of the claimed pharmaceutical composition of slowed down release for the peroral administration, which contains insulin on the carrier-chitosan obtained with the use of the method characterized above.

The parameters of the method proposed for obtaining the composition after the invention are experimentally specified. The essential features of the method to be worded in the independent claim of the invention are necessary for achieving sufficient stability for the carrier-chitosan sol, obtaining the stable mixed nanosized sol of high homogeneity degree and with its ability to have been transformed into gel state up to obtaining a stable and highly structured xerogel transformable into nanosized particles at keeping activity of the operating agent. While applying the obtained xerogel particles, it is possible to produce medicinal forms by means of known methods, both in the form of tablets and pills, and in the form of capsules as well.

Further there exposed the concrete examples of the invention realization.

### Example 1

To obtain the positively charged chitosan sol, there used the chitosan chlorohydrate particles having molecular mass of 100 kDa, deacetylation degree up to 87%, the average particle size of 300 nanometers and zeta-potential +50 mV.

The chosen chitosan particles are intensively agitated with acetate buffered solution (pH=4.0) prepared using bidistilled water, providing for the mol ratio of hydrogen ion (H⁺):glucosamine chitosan link (GlcN) equal to (0.5-1:1. The number of positively charged centers on chitosan surface (positive charge density) depends on degree of protonation of chitosan amino groups. The sol particle zeta-potential has made +50 mV.

Na-insulin is obtained with the use of STRAIN ESCHERICHIA COLI XLI-BLUE/PINSR as PREPROINSULIN PRODUCENT according to patents RU 2148642, 2000 and UA 24452, 2003.

50 ml of the obtained positively charged chitosan sol of 10 mg/ml concentration are mixed with 50 ml of the colloidal solution of negatively charged Na-insulin suspended in the phosphatic buffered solution with pH=9.0, at insulin concentration of 5 mg/ml, that is at insulin:chitosan mass ratio equal to 0.5:1. The pH value of the mixed sol is brought up to 6.0 and kept under such a condition for 10 minutes to obtain the stable electrically neutral gel.

The obtained gel is subjected to the process of dehydration by sublimating drying resulted in obtaining xerogel with the particle size of 10 to 50 mkm.

As a result, there is produced a pharmaceutical composition, which is an insulin and chitosan complex with insulin amount of 190 IU/ml. The above said composition possesses an effect of the slowed down release, and it is recommended for peroral administration.

### Example 2

The method is performed with the use of the positively charged chitosan acetate sol having molecular mass of 80 kDa, deacetylation degree of 85%, and the average particle size of 400 nanometers, which are dispersed in the acetate buffer, the pH value of which makes 3.5.

The negatively charged crystalline particles of human biosynthetic insulin sized of 800 to 1200 nanometers are introduced into chitosan sol at intensive agitation and at insulin:chitosan mass ratio equal to 0.2:1. After that there is added sodium alginate in amount of 5% by mass, then there is added alkali in amount, which is sufficient to obtain the pH value of 5.5, and the process is followed with further agitation within 3 minutes. The obtained electrically neutral gel is subjected to pulverization drying. As a result, there are produced solid particles of the insulin and chitosan complex sized of 50 to 100 mkm.

### Example 3

The method is performed as in example 1 with the use of the positively charged chitosan glutamate sol having molecular mass of 120 kDa, deacetylation degree of 89%, the average particle size of 200 nanometers, which are dispersed in the acetate buffer, the pH value of which makes 4.5. The sol particle zeta-potential has made +35 mV.

The colloid solution of pork insulin in the phosphatic buffer with pH 9.0 is supplied for mixing with the sol at insulin:chitosan mass ratio equal to 0.8:1. After achieving the pH value of 6.5 for the mixed sol and keeping the system under such a condition up to bringing it to the state when it is possible to obtain electrically neutral and stable gel, the product is crushed with the use of a ball-valve mill and dried up. As a result, there is produced an insulin and chitosan complex with insulin amount of 220 IU/ml and particle size of 10 to 100 mkm. The above said complex possesses an effect of slowed down release (of active agent).

The obtained pharmaceutical composition has been checked up on mice.

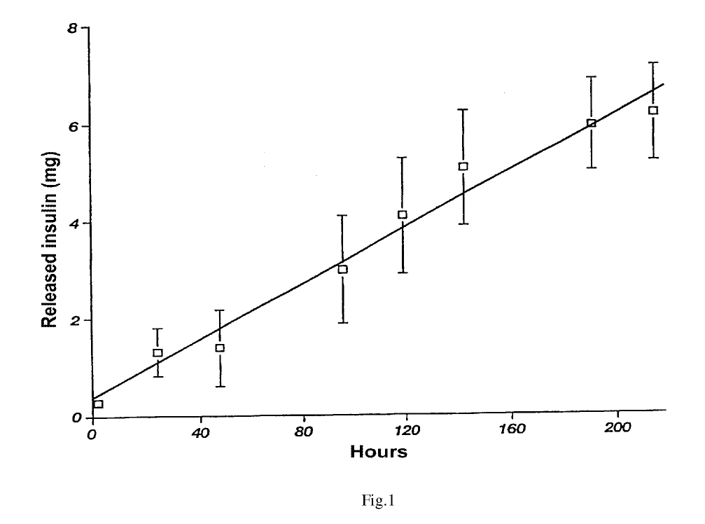

Monitoring kinetics of insulin releasing from the obtained pharmaceutical composition is performed by means of a highly effective liquid and reverse-phase chromatography with gradient elution. Data on continuous release of insulin are presented in FIG. 1 in the form of graphic dependence of insulin amount on time.

The results of the analysis of the inventive solutions have exposed the high efficiency of the insulin-chitosan complex for peroral administration produced according to the claimed method. The obtained agent has exposed the effective controlled release of insulin during 7-8 days.

1. A method for producing a pharmaceutical composition comprising:
providing insulin on a polysaccharide carrier and mixing mixing initial ingredients;
supplying, for mixing, positively charged chitosan sol with pH of 3.5 to 4.5 and negatively charged zinc free insulin, which is taken in the form of a colloidal solution or in the form of nanosized crystalline particles;
bringing the pH of the mixed sol to a value of 5.5 to 6.5;
producing a gel and dehydrating the gel to obtain solid particles, the size of which ranges from 10 to 100 mkm.

2. The method according to claim 1, wherein the gel is produced at a presence of sodium alginate in the mixed sol in the amount not exceeding 5% of masses, if calculated per solid substance.

3. The method according to claim 1, further comprising mixing the initial ingredients based on the results of calculation of an insulin-chitosan mass ratio in the mixed sol to be equal to (0.2-0.8):1 under a condition of the mixed sol electrical neutrality.

4. The method according to claim 1, further comprising supplying for mixing positively charged chitosan sol in a buffered acetate solution with the size of chitosan particles of 200 to 400 nanometers.

5. The method according to claim 1, further comprising supplying for mixing positively charged chitosan sol in the form of its chlorohydrate having molecular mass of 80 to 120 kDa and deacetylation degree of 85 to 89%.

6. The method according to claim 1, further comprising supplying for mixing the insulin in the form of crystalline particles, the sizes of which are ranged from 800 to 1200 nanometers.

7. The method according to claim 1, further comprising supplying for mixing a colloidal solution of insulin in the phosphatic buffer with pH of 8.0 to 9.0.

8. The method according to claim 1, further comprising supplying for mixing chromatographically cleaned gene-engineered insulin obtained with the use of STRAIN ESCHERICHIA COLI XLI-BLUE/PINSR as PREPROINSULIN PRODUCENT.

9. A pharmaceutical composition of slowed down release for peroral administration comprising insulin on a carrier-chitosan, wherein the pharmaceutical composition is obtained using the method comprising providing insulin on a polysaccharide carrier and mixing mixing initial ingredients; supplying, for mixing, positively charged chitosan sol with pH of 3.5 to 4.5 and negatively charged zinc free insulin, which is taken in the form of a colloidal solution or in the form of nanosized crystalline particles; bringing the pH of the mixed sol to a value of 5.5 to 6.5; producing a gel and dehydrating the gel to obtain solid particles, the size of which ranges from 10 to 100 mkm.

10. The pharmaceutical composition of claim 9, wherein the gel is produced at a presence of sodium alginate in the mixed sol in the amount not exceeding 5% of masses, if calculated per solid substance.

11. The pharmaceutical composition of claim 9, wherein mixing the initial ingredients occurs based on the results of calculation of an insulin-chitosan mass ratio in the mixed sol to be equal to (0.2-0.8):1 under a condition of the mixed sol electrical neutrality.

12. The pharmaceutical composition of claim 9, further supplying for mixing positively charged chitosan sol in a buffered acetate solution with the size of chitosan particles of 200 to 400 nanometers.

13. The pharmaceutical composition of claim 9, further supplying for mixing positively charged chitosan sol in the form of its chlorohydrate having molecular mass of 80 to 120 kDa and deacetylation degree of 85 to 89%.

14. The pharmaceutical composition of claim 9, further supplying for mixing the insulin in the form of crystalline particles, the sizes of which are ranged from 800 to 1200 nanometers.

15. The pharmaceutical composition of claim 9, further supplying for mixing a colloidal solution of insulin in the phosphatic buffer with pH of 8.0 to 9.0.

16. The pharmaceutical composition of claim 9, further supplying for mixing chromatographically cleaned gene-engineered insulin obtained with the use of STRAIN ESCHERICHIA COLI XLI-BLUE/PINSR as PREPROINSULIN PRODUCENT.
